(19) United States
(12) Patent Application Publication
(10) Pub. No.: US 2024/0266056 A1
(43) Pub. Date: Aug. 8, 2024

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Yohei MOMOKI, Tokyo (JP)

(73) Assignee: FUJIFILM Corporation, Tokyo (JP)

(21) Appl. No.: 18/617,626

(22) Filed: Mar. 26, 2024

Related U.S. Application Data
(63) Continuation of application No. PCT/JP2022/036597, filed on Sep. 29, 2022.

(30) Foreign Application Priority Data
Sep. 30, 2021 (JP) 2021-162033

(51) Int. Cl. G16H 50/20; G06F 40/166; G06T 7/00; G06V 10/25

(52) U.S. Cl. CPC G16H 50/20; G06F 40/166; G06T 7/0012; G06V 10/25

(57) ABSTRACT

An information processing apparatus including at least one processor, wherein the processor is configured to: generate a graph structure represented by a node indicating each of a plurality of pieces of element information used for medical diagnosis and an edge connecting the nodes of the related pieces of element information; and generate a sentence related to the medical diagnosis based on the graph structure.

### CROSS REFERENCE TO RELATED APPLICATIONS

This application is a continuation of International Application No. PCT/JP2022/036597, filed on Sep. 29, 2022, which claims priority from Japanese Patent Application No. 2021-162033, filed on Sep. 30, 2021. The entire disclosure of each of the above applications is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. In addition, image diagnosis is made by analyzing medical images via computer aided detection/diagnosis (CAD) using a discriminator in which learning is performed by deep learning or the like, and detecting and/or diagnosing regions of interest including structures, lesions, and the like included in the medical images. The medical images and analysis results via CAD are transmitted to a terminal of a healthcare professional such as a radiologist who interprets the medical images. The healthcare professional such as a radiologist interprets the medical image by referring to the medical image and analysis result using his or her own terminal and creates an interpretation report.

In addition, various methods have been proposed to support the creation of medical documents such as interpretation reports in order to reduce the burden of the interpretation work of a radiologist. For example, JP2019-153250A discloses a technology for creating a medical document such as an interpretation report based on a keyword input by a radiologist and an analysis result of a medical image. In the technology disclosed in JP2019-153250A, a sentence to be included in the interpretation report is created by using a recurrent neural network trained to generate a sentence from input characters.

For example, JP2006-181137A discloses a technology for generating and presenting support information related to an input medical image by checking medical information input through input means against a medical thesaurus dictionary prepared in advance.

In recent years, as the performance of imaging apparatuses has improved, the amount of information on analysis results obtained from medical images has tended to increase, and therefore the amount of sentences described in medical documents such as interpretation reports has also tended to increase. Therefore, there is a need for a technology that can create appropriate medical documents such as interpretation reports even in a case where there are a large number of medical image analysis results. However, in the related art, in a case of trying to generate complicated and large amounts of sentences, there are cases where the information is omitted or the sentences become redundant.

### SUMMARY

The present disclosure provides an information processing apparatus, an information processing method, and an information processing program capable of supporting creation of medical documents.

According to a first aspect of the present disclosure, there is provided an information processing apparatus comprising at least one processor, in which the processor is configured to: generate a graph structure represented by a node indicating each of a plurality of pieces of element information used for medical diagnosis and an edge connecting the nodes of the related pieces of element information; and generate a sentence related to the medical diagnosis based on the graph structure.

According to a second aspect of the present disclosure, in the first aspect, the element information may be information indicating at least one of a name, a property, a measured value, a position, or an estimated disease name related to a region of interest included in a medical image, or an imaging method, an imaging condition, or an imaging date and time related to imaging of the medical image.

According to a third aspect of the present disclosure, in the second aspect, the region of interest may be at least one of a region of a structure included in the medical image or a region of an abnormal shadow included in the medical image.

According to a fourth aspect of the present disclosure, in any one of the first to third aspects, the processor may be configured to connect the nodes indicating the plurality of pieces of element information regarding the same region of interest included in a medical image with the edge.

According to a fifth aspect of the present disclosure, in any one of the first to fourth aspects, the processor may be configured to connect the nodes indicating the pieces of element information regarding a plurality of different regions of interest included in a medical image with the edge via a node indicating a physical correlation of the plurality of different regions of interest.

According to a sixth aspect of the present disclosure, in any one of the first to fourth aspects, the processor may be configured to connect the nodes indicating the pieces of element information regarding a plurality of different regions of interest included in a medical image with an edge indicating a physical correlation of the plurality of different regions of interest.

According to a seventh aspect of the present disclosure, in any one of the first to sixth aspects, the processor may be configured to connect the nodes indicating the pieces of element information regarding regions of interest included in a plurality of medical images of the same subject captured at different imaging points in time with the edge via a node indicating a change over time in the regions of interest.

According to an eighth aspect of the present disclosure, in any one of the first to sixth aspects, the processor may be configured to connect the nodes indicating the pieces of element information regarding regions of interest included in a plurality of medical images of the same subject captured at different imaging points in time with an edge indicating a change over time in the regions of interest.

According to a ninth aspect of the present disclosure, in any one of the first to eighth aspects, the processor may be configured to: divide a plurality of the nodes and a plurality of the edges included in the graph structure into a plurality of groups; generate a sentence for each group; and generate a sentence related to the medical diagnosis by combining a plurality of the sentences generated for each group.

According to a tenth aspect of the present disclosure, in any one of the first to ninth aspects, the processor may be configured to generate the sentence by inputting the generated graph structure to a trained model that has been trained in advance such that an input is the graph structure and an output is the sentence.

According to an eleventh aspect of the present disclosure, in the tenth aspect, the trained model may be trained using a set of a permutation graph structure in which the node in the graph structure is permuted with a placeholder predetermined for each category of the element information indicated by the node, and a sentence expressed including the placeholder as training data, and the processor may be configured to: generate the permutation graph structure in which the node in the generated graph structure is permuted with the placeholder; generate the sentence expressed including the placeholder by inputting the permutation graph structure to the trained model; and permute the placeholder included in the sentence with a character string indicated by the element information.

According to a twelfth aspect of the present disclosure, in any one of the first to eleventh aspects, the processor may be configured to: acquire a medical image; and generate the element information based on the acquired medical image.

According to a thirteenth aspect of the present disclosure, in any one of the first to twelfth aspects, the information processing apparatus may further comprise an input unit, and the processor may be configured to generate the element information based on information input via the input unit.

According to a fourteenth aspect of the present disclosure, in any one of the first to thirteenth aspects, the processor may be configured to acquire the element information from an external device.

According to a fifteenth aspect of the present disclosure, there is provided an information processing method comprising: generating a graph structure represented by a node indicating each of a plurality of pieces of element information used for medical diagnosis and an edge connecting the nodes of the related pieces of element information; and generating a sentence related to the medical diagnosis based on the graph structure.

According to a sixteenth aspect of the present disclosure, there is provided an information processing program for causing a computer to execute: generating a graph structure represented by a node indicating each of a plurality of pieces of element information used for medical diagnosis and an edge connecting the nodes of the related pieces of element information; and generating a sentence related to the medical diagnosis based on the graph structure.

The information processing apparatus, the information processing method, and the information processing program according to the aspects of the present disclosure can support the creation of medical documents.

### BRIEF DESCRIPTION OF THE DRAWINGS

FIG. 1 is a diagram showing an example of a schematic configuration of an information processing system.

FIG. 2 is a block diagram showing an example of a hardware configuration of an information processing apparatus.

FIG. 3 is a block diagram showing an example of a functional configuration of the information processing apparatus.

FIG. 4 is a diagram showing an example of a dictionary of element information.

FIG. 5 is a diagram showing an example of a dictionary of element information.

FIG. 6 is a diagram showing an example of a dictionary of element information.

FIG. 7 is a diagram showing an example of a dictionary of element information.

FIG. 8 is a diagram showing an example of a dictionary of element information.

FIG. 9 is a diagram showing an example of element information.

FIG. 10 is a diagram showing an example of element information.

FIG. 11 is a diagram showing an example of a graph structure.

FIG. 12 is a diagram showing an example of a graph structure.

FIG. 13 is a diagram showing an example of a graph structure.

FIG. 14 is a diagram showing an example of a permutation graph structure.

FIG. 15 is a diagram showing an example of a graph structure.

FIG. 16 is a diagram showing an example of a graph structure divided into groups.

FIG. 17 is a diagram showing an example of sentences generated for each group.

FIG. 18 is a diagram showing an example of a screen displayed on a display.

FIG. 19 is a flowchart showing an example of information processing.

FIG. 20 is a diagram showing a modification example of the graph structure.

FIG. 21 is a diagram showing a modification example of the graph structure.

FIG. 22 is a diagram showing a modification example of the graph structure.

FIG. 23 is a diagram showing a modification example of the graph structure.

### DETAILED DESCRIPTION

Hereinafter, an exemplary embodiment of the present disclosure will be described with reference to the drawings.

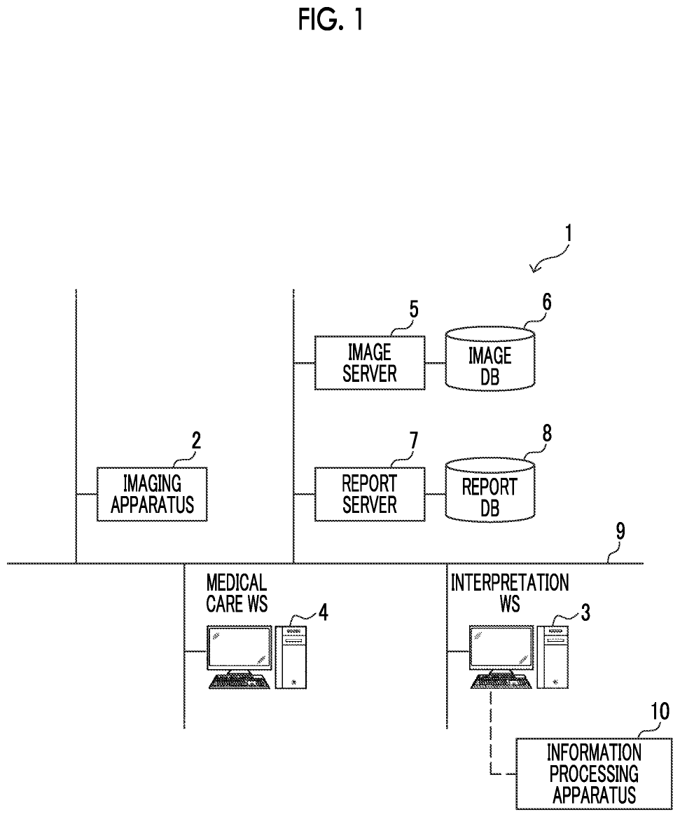

First, a configuration of an information processing system 1 to which an information processing apparatus of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system 1 performs an interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation work station (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc (DVD) or a compact disc read-only memory (CD-ROM), and distributed, and be installed on the computer from the recording medium. In addition, the application program may be stored in, for example, a storage apparatus of a server computer connected to the network 9 or in a network storage in a state in which it can be accessed from the outside, and be downloaded and installed on the computer in response to a request.

The imaging apparatus 2 is an apparatus (modality) that generates a medical image showing a diagnosis target part of the subject by imaging the diagnosis target part. Specifically, examples of the imaging apparatus include a simple X-ray imaging apparatus, a CT apparatus, an MRI apparatus, a positron emission tomography (PET) apparatus, and the like. The medical image generated by the imaging apparatus 2 is transmitted to the image server 5 and is stored in the image DB 6.

The interpretation WS 3 is a computer used by, for example, a healthcare professional such as a radiologist of a radiology department to interpret a medical image and to create an interpretation report, and encompasses an information processing apparatus 10 according to the present exemplary embodiment. In the interpretation WS 3, a viewing request for a medical image to the image server 5, various types of image processing for the medical image received from the image server 5, display of the medical image, and input reception of a sentence regarding the medical image are performed. In the interpretation WS 3, analysis processing for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing device, a display device such as a display, and an input device such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network attached storage (NAS) and a storage area network (SAN).

The image DB 6 is realized by, for example, a storage medium such as a hard disk drive (HDD), a solid-state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, and the like. In addition, the accessory information may include information related to the subject such as the name, age, and gender of the subject.

In a case where the image server 5 receives a request to register a medical image from the imaging apparatus 2, the image server 5 prepares the medical image in a format for a database and registers the medical image in the image DB 6. In addition, in a case where the viewing request from the interpretation WS 3 and the medical care WS 4 is received, the image server 5 searches for a medical image registered in the image DB 6 and transmits the searched for medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered.

Further, in a case where the report server 7 receives a request to register the interpretation report from the interpretation WS 3, the report server 7 prepares the interpretation report in a format for a database and registers the interpretation report in the report DB 8. Further, in a case where the report server 7 receives the viewing request for the interpretation report from the interpretation WS 3 and the medical care WS 4, the report server 7 searches for the interpretation report registered in the report DB 8, and transmits the searched for interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

Next, the information processing apparatus 10 according to the present exemplary embodiment will be described. The information processing apparatus 10 has a function of supporting the creation of a medical document such as an interpretation report based on a medical image captured by the imaging apparatus 2. As described above, the information processing apparatus 10 is encompassed in the interpretation WS 3.

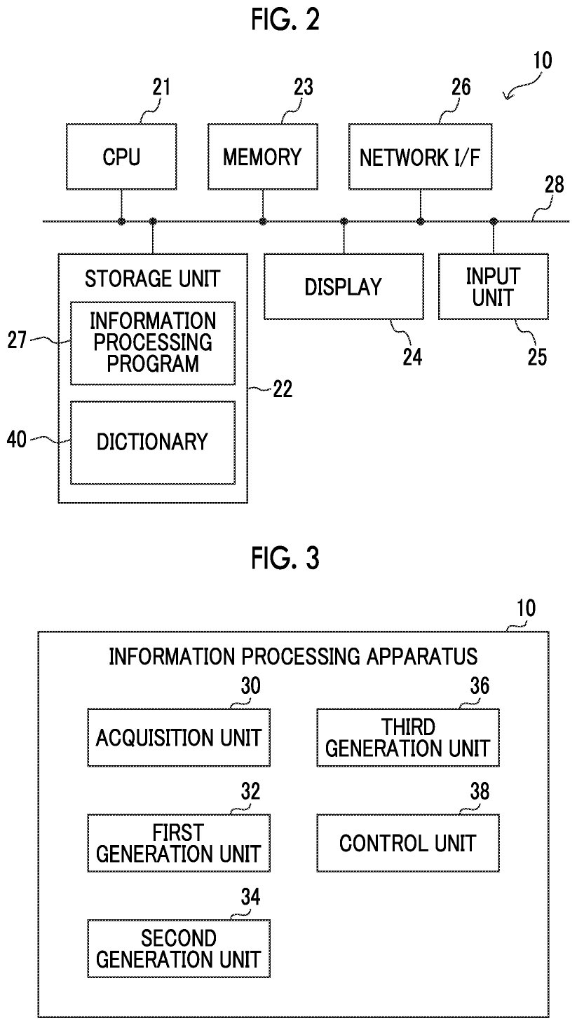

First, with reference to FIG. 2, an example of a hardware configuration of the information processing apparatus 10 according to the present exemplary embodiment will be described. As shown in FIG. 2, the information processing apparatus 10 includes a central processing unit (CPU) 21, a non-volatile storage unit 22, and a memory 23 as a temporary storage area. Further, the information processing apparatus 10 includes a display 24 such as a liquid crystal display, an input unit 25 such as a keyboard and a mouse, and a network interface (I/F) 26. The network I/F 26 is connected to the network 9 and performs wired or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. The storage unit 22 stores an information processing program 27 in the information processing apparatus 10 and a dictionary 40 (details will be described later). The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be appropriately applied.

Next, with reference to FIG. 3, an example of a functional configuration of the information processing apparatus 10 according to the present exemplary embodiment will be described. As shown in FIG. 3, the information processing apparatus 10 includes an acquisition unit 30, a first generation unit 32, a second generation unit 34, a third generation unit 36, and a control unit 38. In a case where the CPU 21 executes the information processing program 27, the CPU 21 functions as the acquisition unit 30, the first generation unit 32, the second generation unit 34, the third generation unit 36, and the control unit 38.

The acquisition unit 30 acquires a medical image to be created as an interpretation report from the image server 5. In the following description, an example will be described in which the medical image acquired by the acquisition unit 30 is a medical image related to lungs.

The first generation unit 32 generates element information used for medical diagnosis based on the medical image acquired by the acquisition unit 30. Specifically, the first generation unit 32 extracts a region of interest including at least one of a region of a structure (for example, organs, tissues, and the like) included in the medical image or a region of an abnormal shadow (for example, the shadow due to a lesion such as a nodule) included in the medical image. For the extraction of the region of interest, for example, a trained model such as a convolutional neural network (CNN), which has been trained in advance to input a medical image and output a region of interest extracted from the medical image, may be used. Further, the first generation unit 32 may extract a region in the medical image designated by a user via the input unit 25 as a region of interest.

Further, the first generation unit 32 generates element information related to the region of interest extracted from the medical image. For the generation of the element information by the first generation unit 32, for example, a trained model such as a CNN, which has been trained in advance to input a region of interest in the medical image and output element information related to the region of interest, may be used.

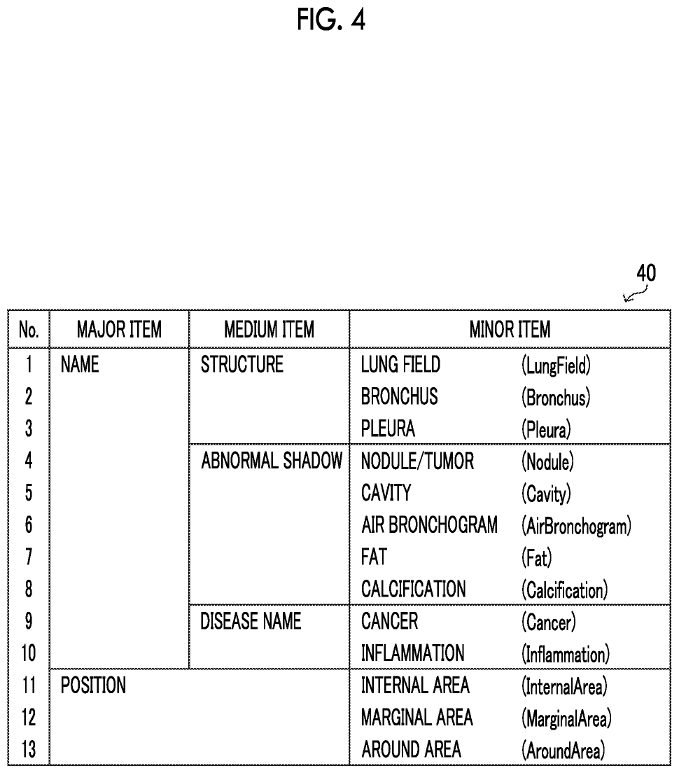

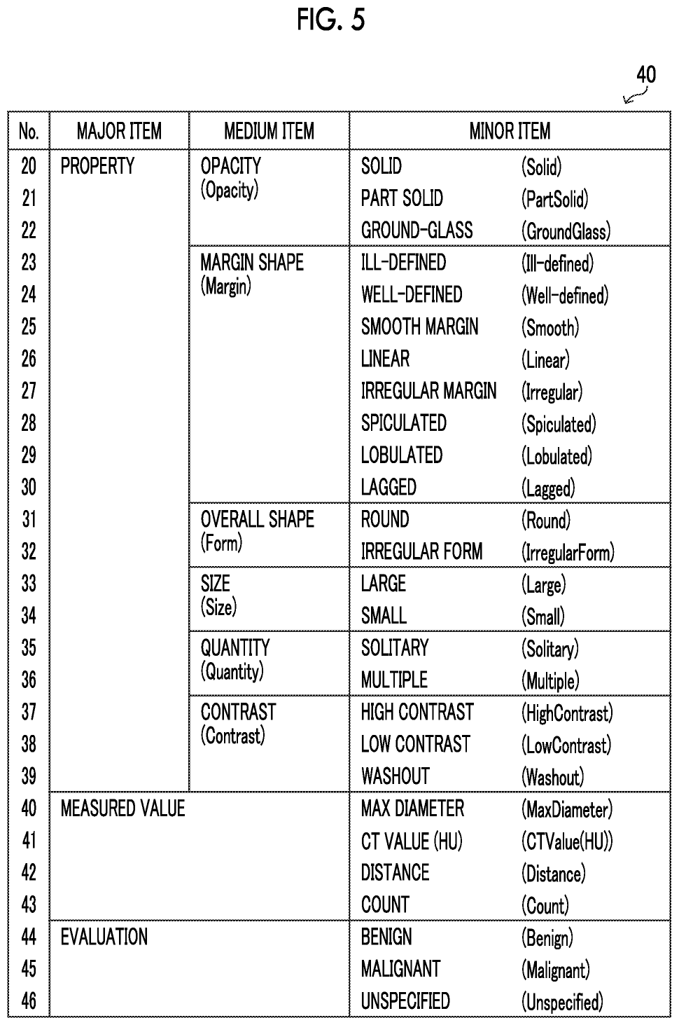

FIGS. 4 to 8 show examples of the dictionary 40 in which element information that can be generated by the first generation unit 32 is predetermined. The dictionary 40 shown in FIGS. 4 to 8 mainly relates to lungs. FIGS. 4 and 5 show element information indicating a name (type), a property, a measured value, a position, and an estimated disease name (including negative or positive evaluation results) regarding the region of interest included in the medical image. The first generation unit 32 may specify at least one of the name (type), property, measured value, position, estimated disease name, or the like of the region of interest extracted from the medical image, and generate the specified information as element information.

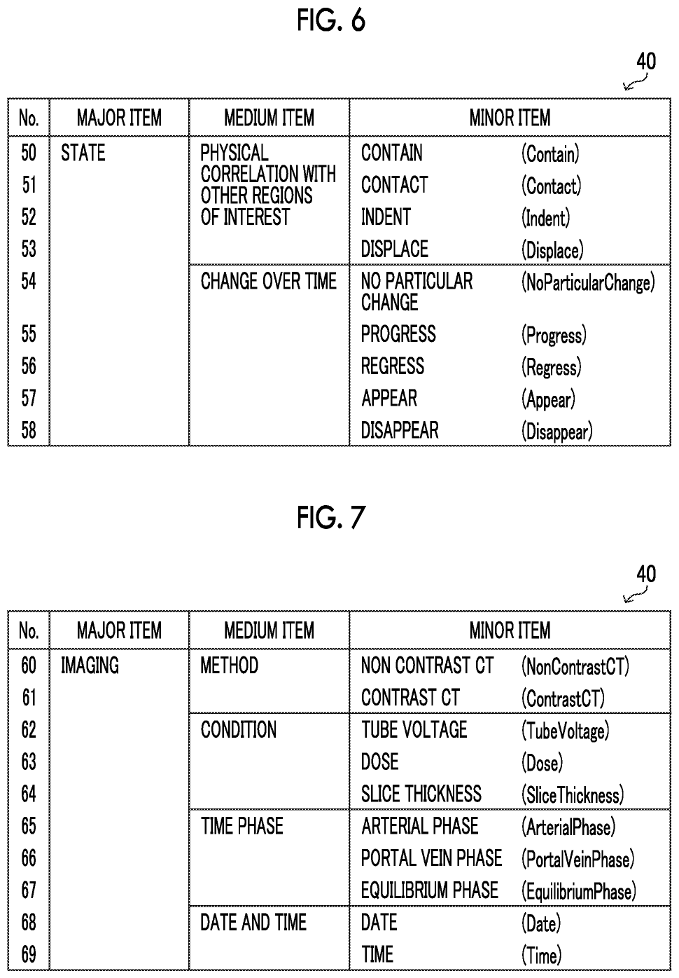

FIG. 6 shows element information indicating a physical correlation of a plurality of different regions of interest included in a medical image. In a case where the medical image includes a plurality of different regions of interest, the first generation unit 32 may specify a physical correlation of the regions of interest and generate the specified information as element information.

Further, FIG. 6 shows element information indicating changes over time in a region of interest included in a medical image. For example, in a case where the acquisition unit 30 acquires a plurality of medical images of the same subject captured at different imaging points in time, the first generation unit 32 may specify changes over time in the region of interest included in each medical image, and generate the specified information as element information.

FIG. 7 shows element information indicating an imaging method, imaging conditions, an imaging time phase, and an imaging date and time regarding imaging of medical images. As described above, each medical image is attached by accessory information including information related to imaging at the time of being registered in the image DB 6. The first generation unit 32 may generate element information based on accessory information attached to a medical image.

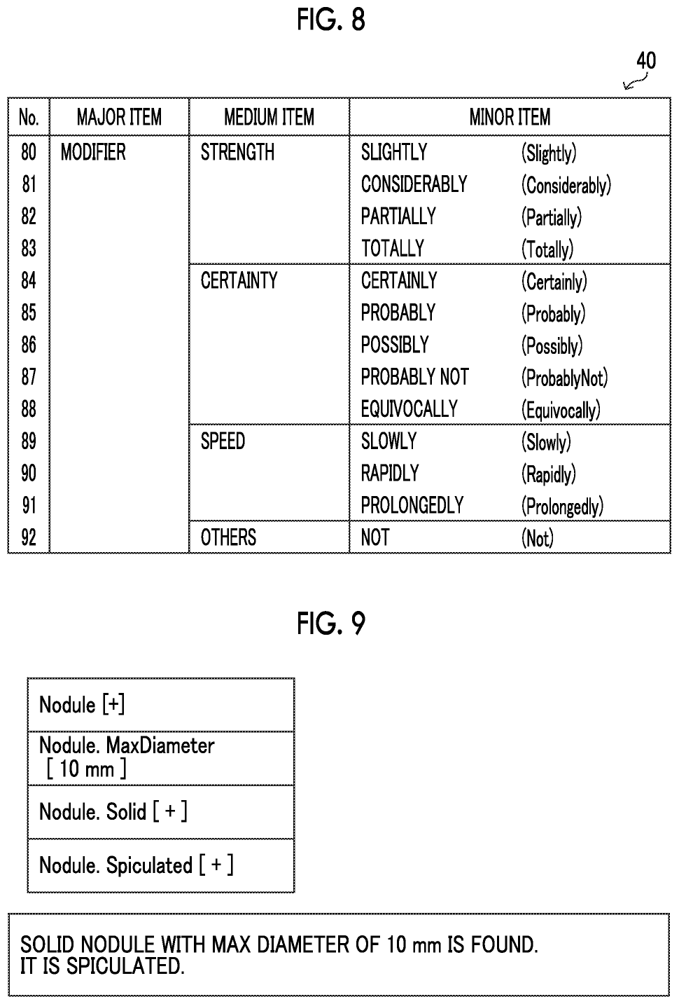

FIG. 8 shows element information that modifies the above element information. The first generation unit 32 may add the element information shown in FIG. 8 to the element information shown in FIGS. 4 to 7. In addition to this, for example, the first generation unit 32 may acquire information included in an examination order and an electronic medical record, information indicating various test results such as a blood test and an infectious disease test, information indicating the result of a health diagnosis, and the like from the external device such as the medical care WS 4, and generate the acquired information as element information as appropriate.

In addition, the first generation unit 32 may generate element information based on the information input via the input unit 25. For example, the first generation unit 32 may check a keyword input by the user via the input unit 25 against the dictionary 40 and select element information corresponding to the keyword. Further, for example, the first generation unit 32 may present the dictionary 40 on the display 24 and receive the designation of the element information by the user.

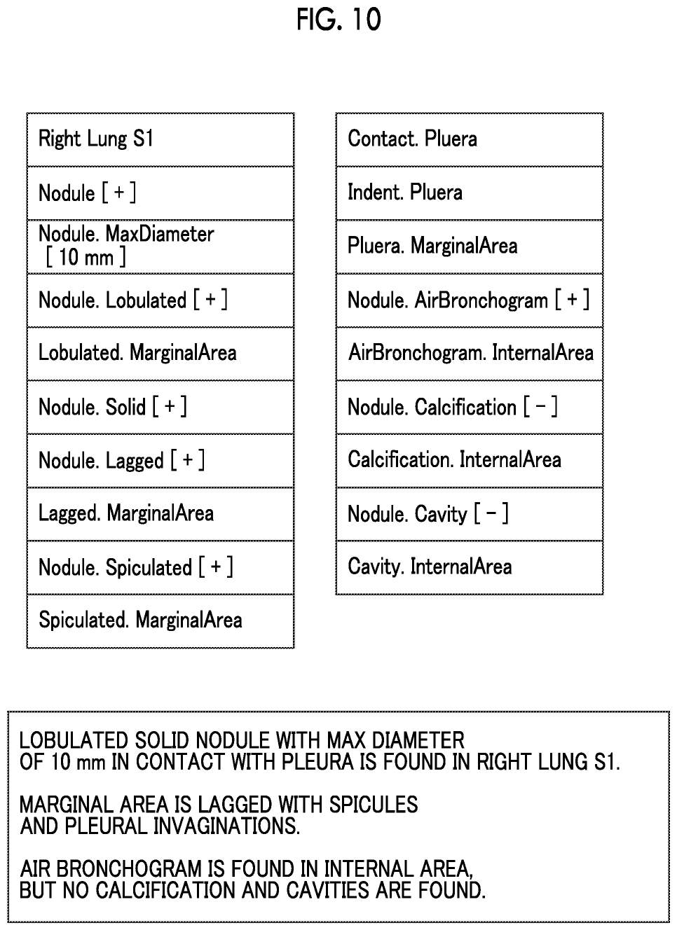

Here, element information generated by the first generation unit 32 will be described with reference to FIGS. 9 and 10. In FIGS. 9 and 10, element information generated by the first generation unit 32 and sentences to be generated based on the element information are described. In FIGS. 9 and 10, element information is written in English as shown in the dictionary 40 of FIGS. 4 to 8. The properties and positions are written after the name of the corresponding region of interest. Measured values and negative (minus) or positive (plus) evaluation results are written in [ ].

As shown in FIG. 9, in a case where the amount of element information is relatively small, it is easy to generate sentences appropriately even in a case where the sentences are directly generated from the element information. This is because, for example, in a case of generating sentences using a trained model that inputs element information and outputs sentences, it is easy to prepare supervised training data containing similar combinations of element information in the learning phase of the model. Furthermore, for example, even in a case where a sentence is generated by embedding element information in a template with a predetermined sentence structure, it is easy to prepare a template, and it is thus easy to generate an appropriate sentence.

On the other hand, as shown in FIG. 10, in a case where the amount of element information is relatively large and sentences are generated directly from the element information, information may be omitted, sentences may become redundant, or element information regarding a plurality of regions of interest may be replaced. This is because, for example, in the learning phase of a model that generates sentences from the above element information, it is difficult to prepare supervised training data that covers combinations of similar element information. Further, for example, even in a case where sentences are generated by embedding element information in a template with a predetermined sentence structure, the variations in templates become enormous, making it difficult to prepare a template that covers all combinations of element information.

Therefore, the second generation unit 34 organizes the element information generated by the first generation unit 32 to prepare for facilitating generation of appropriate sentences. Specifically, the second generation unit 34 generates a graph structure represented by a node indicating each of a plurality of pieces of element information regarding the medical image generated by the first generation unit 32 and an edge connecting the nodes of the related pieces of element information.

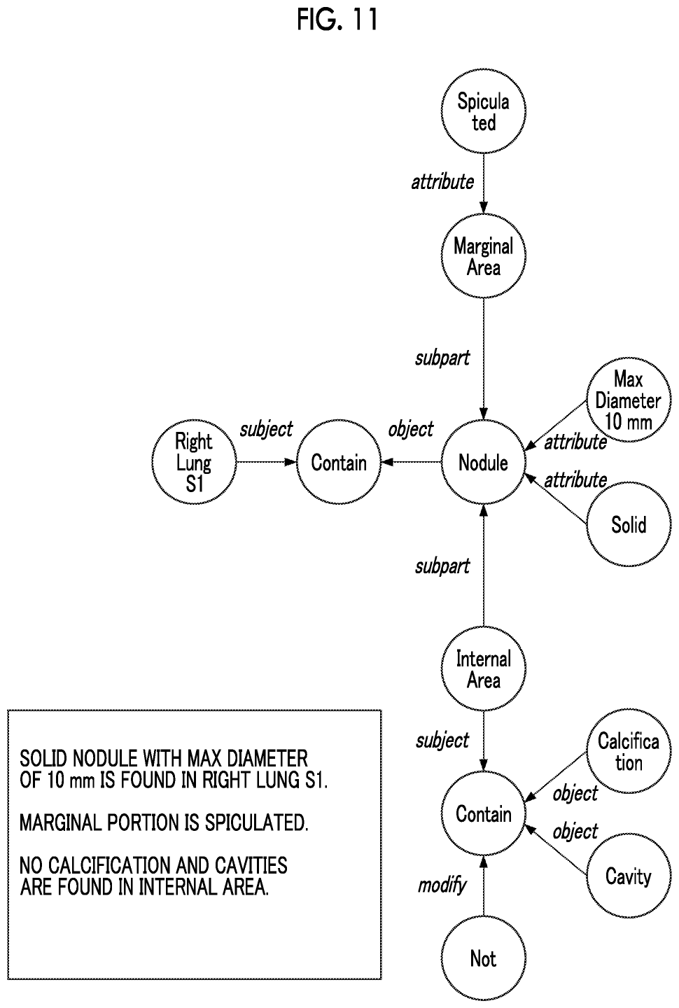

FIGS. 11 to 13 show graph structures generated by the second generation unit 34 and sentences to be generated based on the graph structures. In FIGS. 11 to 13, element information is written in English as shown in the dictionary 40 in FIGS. 4 to 8. FIGS. 11 to 13 are so-called directed graphs in which nodes are represented by circles and edges are represented by arrows, and the nodes of the related element information are connected by edges. Also, the meaning of edge is shown in italics. In the following description, the nodes and edges shown in each drawing are enclosed in brackets [ ].

As shown in FIG. 11, the second generation unit 34 may connect nodes indicating a plurality of pieces of element information regarding the same region of interest included in the medical image with the edge. For example, in a case where a “solid nodule” is included in a medical image, a region in the medical image that is a basis for generating the element information of “nodule” and a region in the medical image that is a basis for generating the element information of “solid” are the same. Therefore, the second generation unit 34 connects the [Nodule] node and the [Solid] node with edges, as shown in FIG. 11.

Further, as shown in FIG. 11, the second generation unit 34 may connect nodes indicating element information regarding each of a plurality of different regions of interest included in the medical image with the edge via a node indicating a physical correlation of the plurality of different regions of interest (see FIG. 6). For example, it is assumed that there is a nodule in a right lung S1 (S1 indicates a lung area) in a medical image. In this case, a region of interest that contains “Right Lung S1” as a structure and a region of interest that contains “Nodule” as an abnormal shadow within the region of interest are extracted from the medical image. Therefore, as shown in FIG. 11, the second generation unit 34 connects the [Right Lung S1] node and the [Nodule] node with the edge via a [Contain] node indicating a physical correlation.

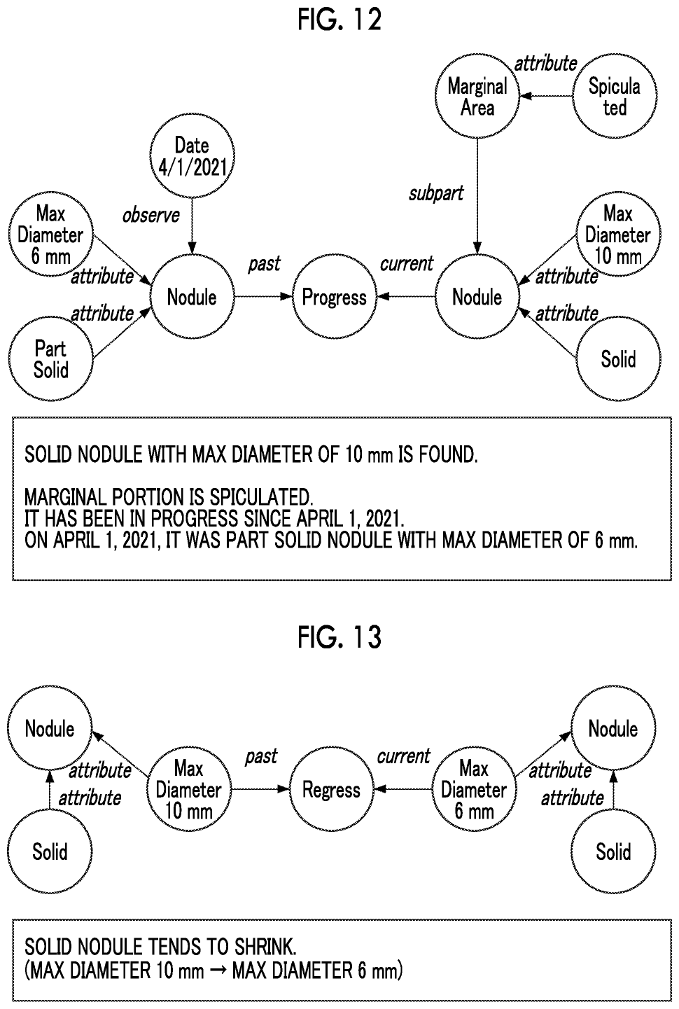

As shown in FIGS. 12 and 13, the second generation unit 34 may connect nodes indicating element information regarding each region of interest included in each of a plurality of medical images of the same subject captured at different imaging points in time with the edge via a node indicating a change over time in the region of interest (see FIG. 6). Nodes indicating changes over time are connected to other nodes with edges representing past and/or current.

For example, as a result of re-imaging a subject that has been imaged in the past and generating element information based on each of the past and current medical images, it is assumed that it is found that the max diameter of the same nodule is increasing (Progress). In this case, as shown in FIG. 12, the second generation unit 34 connects a [Nodule] node on the [past] side and a [Nodule] node on the [current] side via the [Progress] node indicating a change over time with edges.

Further, for example, in a case of generating a graph structure by paying attention to changes in the max diameter, the second generation unit 34 may generate a graph structure as shown in FIG. 13. FIG. 13 shows the graph structure in a case where it is found that the max diameter of the same nodule is decreasing (Regress) as a result of generating element information based on each of past and current medical images. As shown in FIG. 13, the second generation unit 34 may connect [Max Diameter] on the [past] side and [current] side with an edge via a [Regress] node indicating a change over time.

The third generation unit 36 generates sentences related to medical diagnosis based on the graph structure generated by the second generation unit 34. Specifically, the third generation unit 36 may generate a sentence by inputting the graph structure generated by the second generation unit 34 to a trained model M (not shown) such as a CNN, which has been trained in advance such that the input is a graph structure and the output is a sentence. The trained model M is an example of a trained model of the present disclosure.

### Use of Placeholders

Incidentally, as shown in FIGS. 4 to 8, a large amount of element information is recorded in the dictionary 40. On the other hand, for example, regarding a property, pieces of element information of the same category, such as “solid” and “part solid” which are element information regarding opacity, are often used in the same way in sentences. Furthermore, for example, measured values such as “max diameter” are often used in the same way in sentences even in a case where the numerical values are different. Further, for example, the lung area “right lung S1” illustrated in FIG. 11 is often used in the same way in sentences even for other lung areas.

Therefore, in the learning phase of the model M that generates sentences from the above graph structure, it is possible to efficiently train the model M by replacing each piece of element information that is used in the same way in the sentences with placeholders. Specifically, the model M may be trained using a set of a permutation graph structure in which a node in the graph structure is permuted with a placeholder predetermined for each category of element information indicated by the node, and a sentence expressed including the placeholder as training data.

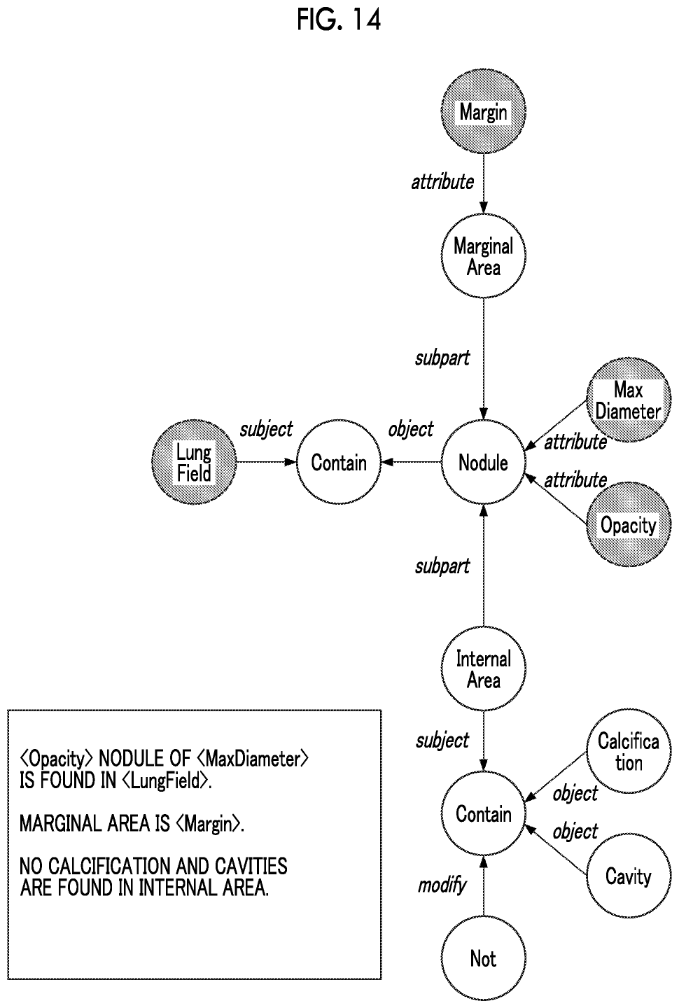

FIG. 14 shows a permutation graph structure in which some of the nodes in the graph structure shown in FIG. 11 are permuted with placeholders, and a sentence expressed including the placeholder. In FIG. 14, the background color of the node permuted with the placeholder is changed to gray, and the frame line is changed to a broken line. In FIG. 14, the [Right Lung S1] node shown as a specific lung area in FIG. 11 is permuted with the [Lung Field] node, and the [Max Diameter 10 mm] shown as a specific max diameter is permuted with a [Max Diameter] node. Furthermore, the [Solid] node is permuted with an [Opacity] node of the higher-level item, and a [Spiculated] node is permuted with a [Margin] node of the higher-level item (see FIG. 5). The corresponding character strings for sentences are also permuted with placeholders. By using such a set of a permutation graph structure and sentences as training data, it is possible to create a model M that can handle more element information with less training data.

Furthermore, in a case where the third generation unit 36 generates a sentence from a graph structure using the trained model M (operation phase), it is also possible to use a permutation graph structure. Specifically, the third generation unit 36 may generate a permutation graph structure in which nodes in the graph structure generated by the second generation unit 34 are permuted with placeholders, and input the permutation graph structure to the trained model M, thereby generating a sentence expressed including placeholder. For example, in a case where the second generation unit 34 generates the graph structure shown in FIG. 11, the third generation unit 36 converts the graph structure shown in FIG. 11 into the permutation graph structure shown in FIG. 14, and inputs the converted permutation graph structure to the trained model M, thereby generating a sentence expressed including a placeholder.

After that, the third generation unit 36 permutes the placeholder included in the sentence expressed including the placeholder with the character string indicated by the element information, and generates the final sentence. For example, the sentence shown in FIG. 11 is generated as the final sentence by embedding the information indicated by the node before permutation into the placeholder part of the sentence expressed including the placeholders shown in FIG. 14. According to such a form, the accuracy of generated sentences can be improved.

### Division of Graph Structure

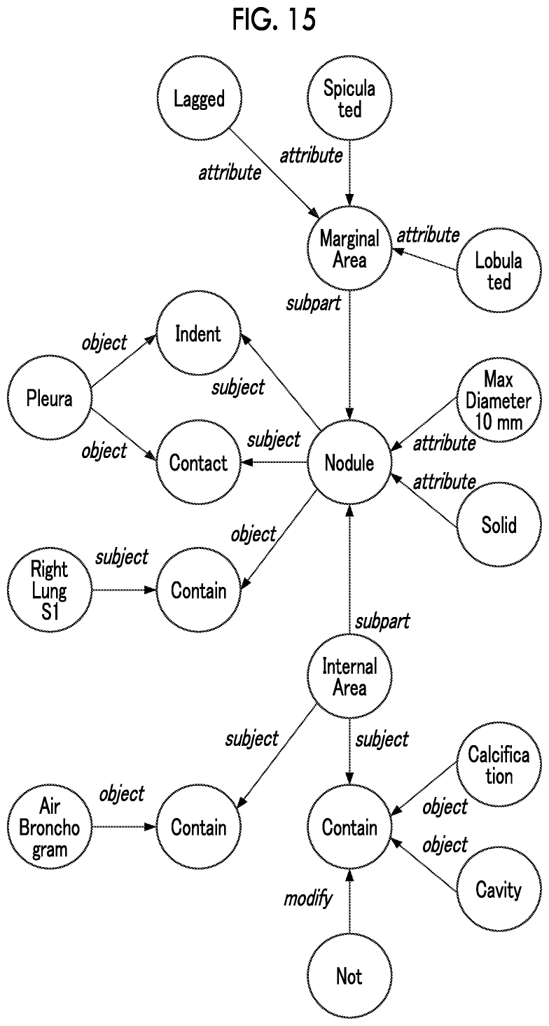

FIG. 15 shows an example of a more complicated graph structure. FIG. 15 is a graph structure corresponding to the sentences in FIG. 10. In a case where the graph structure is complicated, it is preferable that the third generation unit 36 divides a plurality of nodes and a plurality of edges included in the graph structure into a plurality of groups, and generates a sentence for each group. Specifically, the third generation unit 36 may generate a sentence for each group by inputting the divided groups to the trained model M.

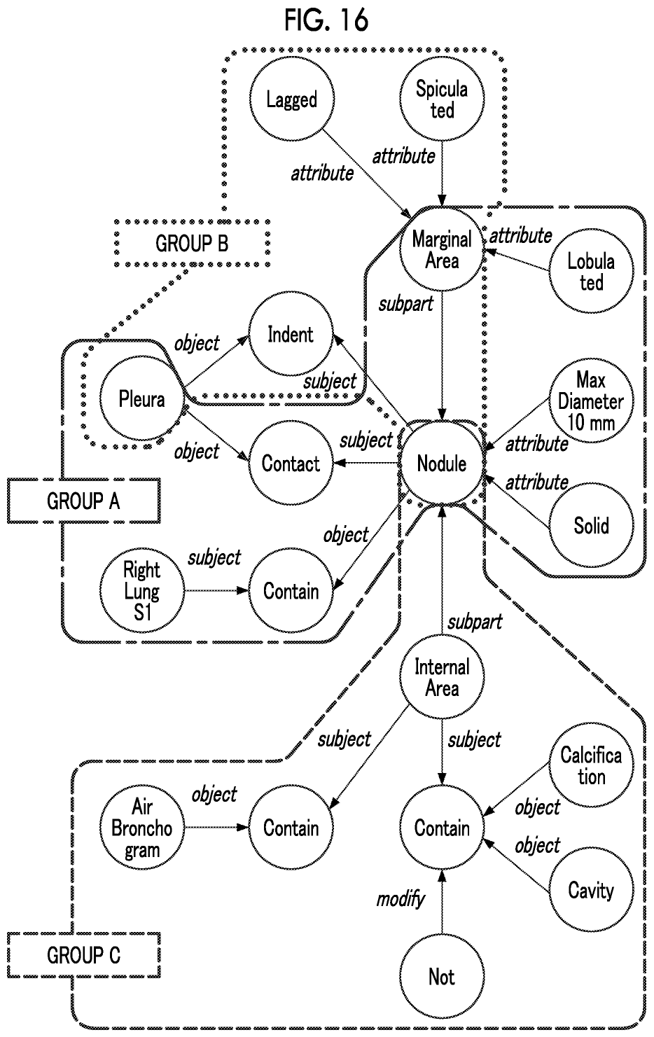

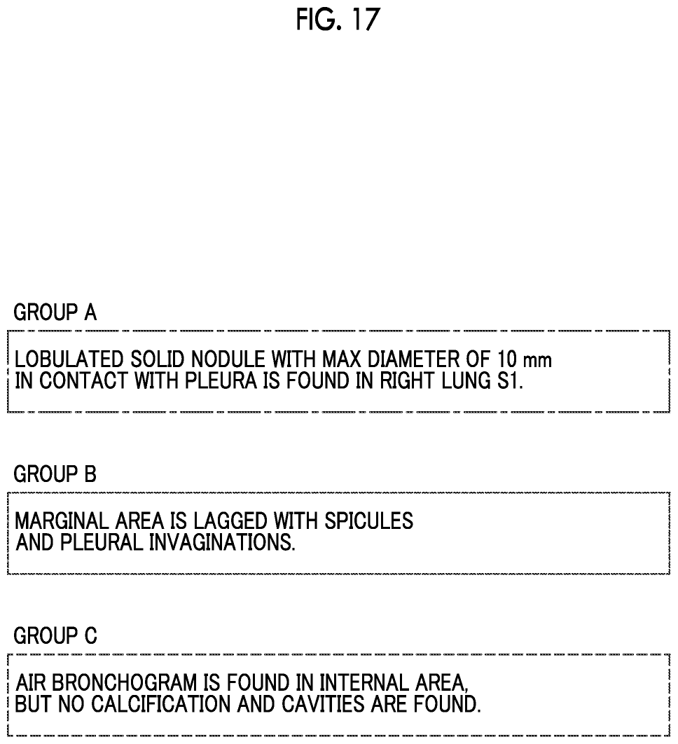

FIG. 16 shows an example in which the graph structure in FIG. 15 is divided into three groups: group A (indicated by an alternated long and short dash line), group B (indicated by a dotted line), and group C (indicated by a broken line). In addition, FIG. 17 shows an example of sentences generated for each of groups A to C in FIG. 16.

For example, a trained model such as a CNN, which is trained in advance to input a graph structure and output a plurality of groups divided from the graph structure, may be used to divide the groups using the third generation unit 36. This trained model is, for example, a model trained using a set of the complicated graph structure as shown in FIG. 15 and the plurality of sentences corresponding to the complicated graph structure as shown in FIG. 17 as training data. In other words, the trained model is a model that has learned a method of dividing a complicated graph structure into groups from a plurality of corresponding sentences.

After generating sentences for each group, the third generation unit 36 combines the plurality of sentences generated for each group to generate a sentence related to medical diagnosis. In this way, even in a case where the graph structure is complicated, by dividing the complicated graph structure into groups and generating sentences, each sentence becomes simpler, and therefore the accuracy of the generated sentences can be improved.

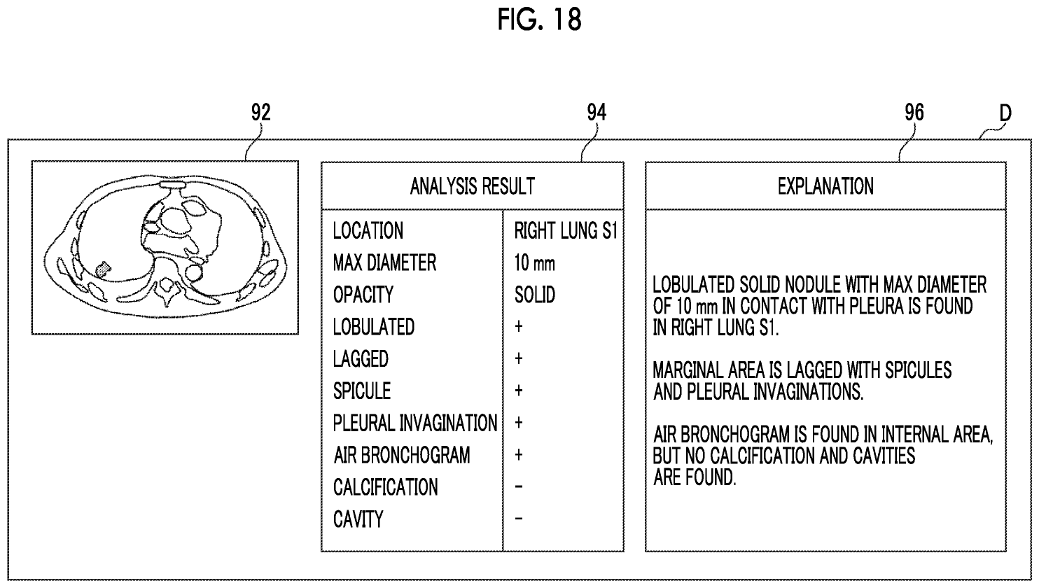

The control unit 38 controls the display 24 to display the sentences related to the medical diagnosis generated by the third generation unit 36. FIG. 18 shows an example of a screen D displayed on the display 24 by the control unit 38. As shown in FIG. 18, the screen D includes a region 92 where the medical image acquired by the acquisition unit 30 is displayed, a region 94 where the element information generated by the first generation unit 32 is displayed, and a region 96 where the sentences related to the medical diagnosis generated by the third generation unit 36 are displayed. Note that the element information and sentences shown in FIG. 18 correspond to the graph structures and sentences shown in FIGS. 15 to 17.

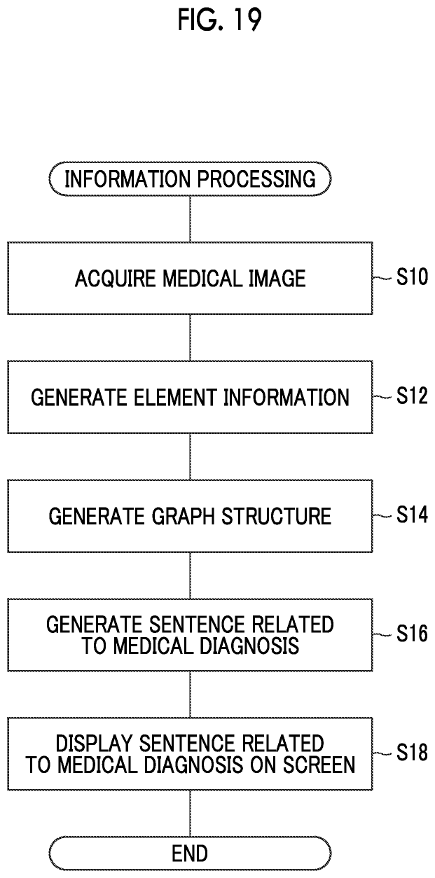

Next, with reference to FIG. 19, operations of the information processing apparatus 10 according to the present exemplary embodiment will be described. In the information processing apparatus 10, as the CPU 21 executes the information processing program 27, information processing shown in FIG. 10 is executed. The information processing is executed, for example, in a case where the user gives an instruction to start execution via the input unit 25.

In Step S10, the acquisition unit 30 acquires a medical image from the image server 5. In Step S12, the first generation unit 32 generates element information based on the medical image acquired in Step S10. In Step S14, the second generation unit 34 generates a graph structure represented by a node indicating each of a plurality of pieces of element information regarding the medical image generated in Step S12 and an edge connecting the nodes of the related pieces of element information. In Step S16, the third generation unit 36 generates a sentence related to medical diagnosis based on the graph structure generated in Step S14. In Step S18, the control unit 38 causes the display 24 to display the screen D including the sentence related to the medical diagnosis generated in Step S16, and ends this information processing.

As described above, the information processing apparatus 10 according to one aspect of the present disclosure comprises at least one processor, and the processor is configured to: generate a graph structure represented by a node indicating each of a plurality of pieces of element information used for medical diagnosis and an edge connecting the nodes of the related pieces of element information; and generate a sentence related to the medical diagnosis based on the graph structure. That is, with the information processing apparatus 10 according to the present exemplary embodiment, by representing element information as a graph structure, it is possible to create sentences including a large amount and a plurality of pieces of element information and to support the creation of medical documents.

Further, in the exemplary embodiment described above, the form in which the first generation unit 32 generates the element information has been described, but the present disclosure is not limited thereto. For example, in a case of creating an interpretation report by generating element information in advance by an external device having the same function as the first generation unit 32 that generates element information based on a medical image, the acquisition unit 30 may be configured to acquire element information from the external device.

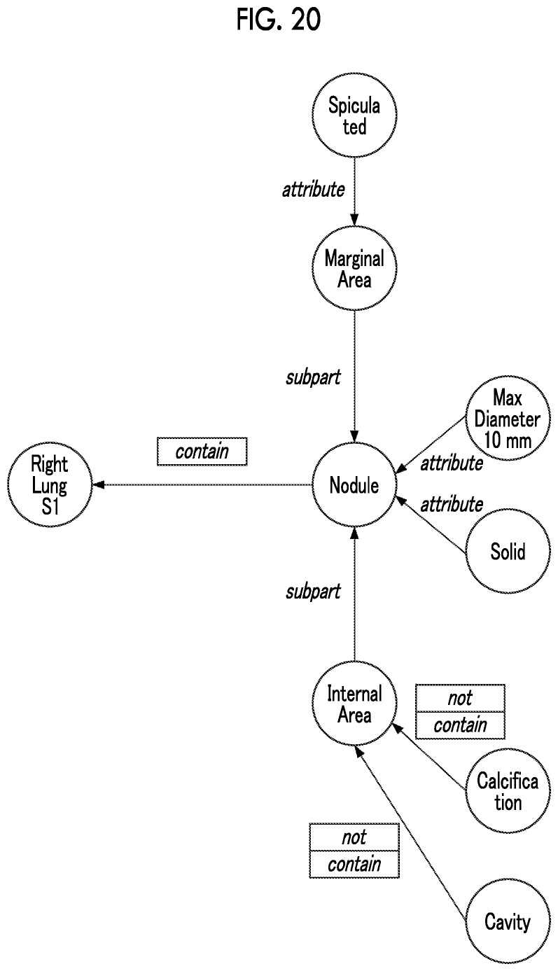

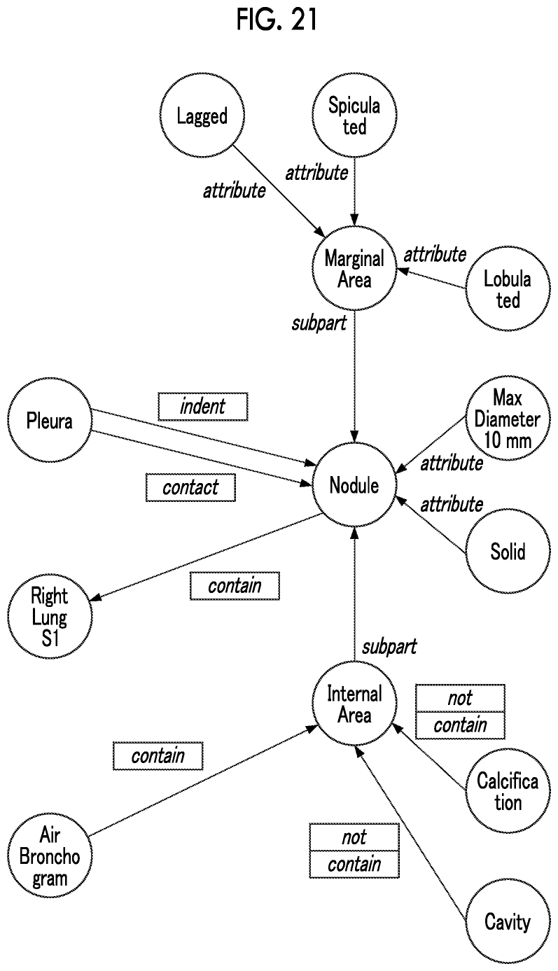

Further, in the exemplary embodiment described above, the form in which the second generation unit 34 represents the physical correlation of a plurality of different regions of interest (see FIG. 6) as nodes has been described, but the present disclosure is not limited thereto. The second generation unit 34 may connect nodes indicating element information regarding each of a plurality of different regions of interest included in a medical image with an edge indicating a physical correlation of the plurality of different regions of interest. As an example, FIGS. 20 and 21 show graph structures in which physical correlations ([Contain], [Indent], and [Contact]) are represented by edges. FIG. 20 corresponds to FIG. 11, and FIG. 21 corresponds to FIG. 15. In FIGS. 20 and 21, edges indicating physical correlations are surrounded by squares. Furthermore, in FIGS. 20 and 21, the modifier [Not] (see FIG. 8) is also shown surrounded by a square as an edge.

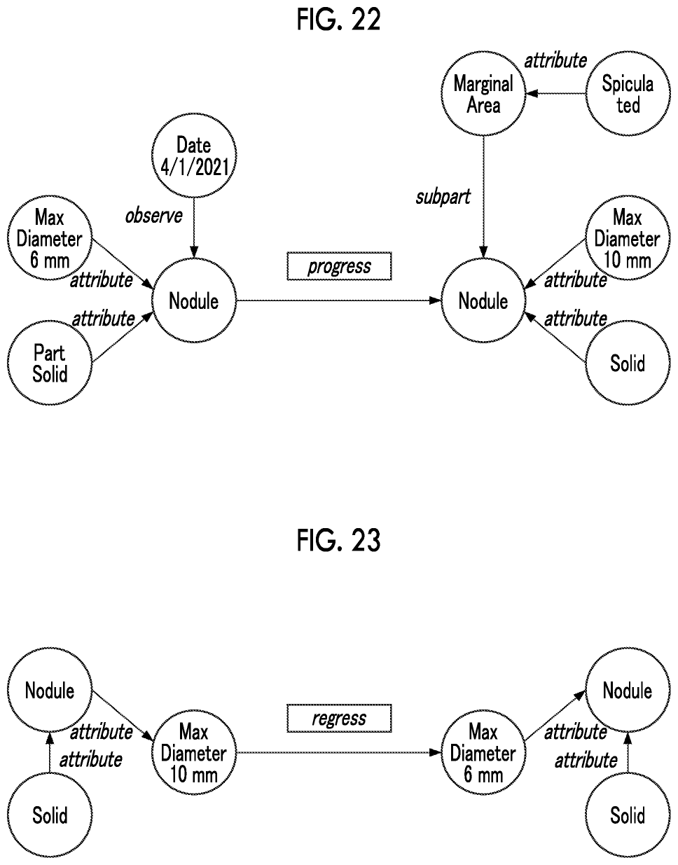

Further, in the exemplary embodiment described above, the form in which the second generation unit 34 represents changes over time in the region of interest (see FIG. 6) as nodes has been described, but the present disclosure is not limited thereto. The second generation unit 34 may connect nodes indicating element information regarding each region of interest included in each of a plurality of medical images of the same subject captured at different imaging points in time with edges indicating changes over time in the region of interest. As an example, FIGS. 22 and 23 show graph structures in which changes over time ([Progress] and [Regress]) are represented by edges. FIG. 22 corresponds to FIG. 12, and FIG. 23 corresponds to FIG. 13. In FIGS. 22 and 23, edges showing changes over time are surrounded by squares.

Further, in the exemplary embodiment described above, the graph structure represented by nodes and edges has been described using a diagram, but the method of expressing the graph structure is not limited to the diagram. The graph structure represented by nodes and edges can also be expressed using techniques such as a resource description framework (RDF) and an adjacency matrix, for example. That is, the technology of the present disclosure is applicable not only to diagrams but also to graph structures expressed by RDFs, adjacency matrices, and the like.

In the above exemplary embodiment, for example, as hardware structures of processing units that execute various kinds of processing, such as the acquisition unit 30, the first generation unit 32, the second generation unit 34, the third generation unit 36, and the control unit 38, various processors shown below can be used. As described above, the various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (program).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system-on-chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above exemplary embodiment, the information processing program 27 is described as being stored (installed) in the storage unit 22 in advance; however, the present disclosure is not limited thereto. The information processing program 27 may be provided in a form recorded in a recording medium such as a compact disc read-only memory (CD-ROM), a digital versatile disc read-only memory (DVD-ROM), and a universal serial bus (USB) memory. In addition, the information processing program 27 may be configured to be downloaded from an external device via a network. Further, the technology of the present disclosure extends to a storage medium for storing the information processing program non-transitorily in addition to the information processing program.

The technology of the present disclosure can be appropriately combined with the above-described exemplary embodiment. The described contents and illustrated contents shown above are detailed descriptions of the parts related to the technology of the present disclosure, and are merely an example of the technology of the present disclosure. For example, the above description of the configuration, function, operation, and effect is an example of the configuration, function, operation, and effect of the parts related to the technology of the present disclosure. Therefore, needless to say, unnecessary parts may be deleted, new elements may be added, or replacements may be made to the described contents and illustrated contents shown above within a range that does not deviate from the gist of the technology of the present disclosure.

The disclosure of JP2021-162033 filed on Sep. 30, 2021 is incorporated herein by reference in its entirety. All documents, patent applications, and technical standards described in the present specification are incorporated in the present specification by reference to the same extent as in a case where each of the documents, patent applications, technical standards are specifically and individually indicated to be incorporated by reference.

What is claimed is:

1. An information processing apparatus comprising at least one processor, wherein the processor is configured to:
generate a graph structure represented by a node indicating each of a plurality of pieces of element information used for medical diagnosis and an edge connecting the nodes of the related pieces of element information; and
generate a sentence related to the medical diagnosis based on the graph structure.

2. The information processing apparatus according to claim 1, wherein the element information is information indicating at least one of a name, a property, a measured value, a position, or an estimated disease name related to a region of interest included in a medical image, or an imaging method, an imaging condition, or an imaging date and time related to imaging of the medical image.

3. The information processing apparatus according to claim 2, wherein the region of interest is at least one of a region of a structure included in the medical image or a region of an abnormal shadow included in the medical image.

4. The information processing apparatus according to claim 1, wherein the processor is configured to connect the nodes indicating the plurality of pieces of element information regarding the same region of interest included in a medical image with the edge.

5. The information processing apparatus according to claim 1, wherein the processor is configured to connect the nodes indicating the pieces of element information regarding a plurality of different regions of interest included in a medical image with the edge via a node indicating a physical correlation of the plurality of different regions of interest.

6. The information processing apparatus according to claim 1, wherein the processor is configured to connect the nodes indicating the pieces of element information regarding a plurality of different regions of interest included in a medical image with an edge indicating a physical correlation of the plurality of different regions of interest.

7. The information processing apparatus according to claim 1, wherein the processor is configured to connect the nodes indicating the pieces of element information regarding regions of interest included in a plurality of medical images of the same subject captured at different imaging points in time with the edge via a node indicating a change over time in the regions of interest.

8. The information processing apparatus according to claim 1, wherein the processor is configured to connect the nodes indicating the pieces of element information regarding regions of interest included in a plurality of medical images of the same subject captured at different imaging points in time with an edge indicating a change over time in the regions of interest.

9. The information processing apparatus according to claim 1, wherein the processor is configured to:
divide a plurality of the nodes and a plurality of the edges included in the graph structure into a plurality of groups;
generate a sentence for each group; and
generate a sentence related to the medical diagnosis by combining a plurality of the sentences generated for each group.

10. The information processing apparatus according to claim 1, wherein the processor is configured to generate the sentence by inputting the generated graph structure to a trained model that has been trained in advance such that an input is the graph structure and an output is the sentence.

11. The information processing apparatus according to claim 10, wherein:
the trained model is trained using a set of a permutation graph structure in which the node in the graph structure is permuted with a placeholder predetermined for each category of the element information indicated by the node, and a sentence expressed including the placeholder as training data, and
the processor is configured to:
generate the permutation graph structure in which the node in the generated graph structure is permuted with the placeholder;
generate the sentence expressed including the placeholder by inputting the permutation graph structure to the trained model; and
permute the placeholder included in the sentence with a character string indicated by the element information.

12. The information processing apparatus according to claim 1, wherein the processor is configured to:
acquire a medical image; and
generate the element information based on the acquired medical image.

13. The information processing apparatus according to claim 1, further comprising an input unit, wherein the processor is configured to generate the element information based on information input via the input unit.

14. The information processing apparatus according to claim 1, wherein the processor is configured to acquire the element information from an external device.

15. An information processing method comprising:
generating a graph structure represented by a node indicating each of a plurality of pieces of element information used for medical diagnosis and an edge connecting the nodes of the related pieces of element information; and
generating a sentence related to the medical diagnosis based on the graph structure.

16. A non-transitory computer-readable storage medium storing an information processing program for causing a computer to execute:
generating a graph structure represented by a node indicating each of a plurality of pieces of element information used for medical diagnosis and an edge connecting the nodes of the related pieces of element information; and
generating a sentence related to the medical diagnosis based on the graph structure.
